(19) United States
(12) Patent Application Publication
(10) Pub. No.: US 2021/0298664 A1
(43) Pub. Date: Sep. 30, 2021

(54) IMPLANTABLE CAPSULE AND GASTROESOPHAGEAL REFLUX DISEASE DETERMINING SYSTEM THEREOF

(71) Applicant: Show-Chwan Memorial Hospital, Changhua City (TW)

(72) Inventors: HSIAO-TING LEE, Lukang Township (TW); SHIH-WEI HUANG, Lukang Township (TW); CHIN-CHENG HSIAO, Lukang Township (TW); KAI-CHE LIU, Lukang Township (TW)

(21) Appl. No.: 17/217,200

(22) Filed: Mar. 30, 2021

Related U.S. Application Data
(60) Provisional application No. 63/002,003, filed on Mar. 30, 2020.

(51) Int. Cl. A61B 5/00; A61B 5/145; A61B 5/0538; A61B 5/07; A61B 5/11

(52) U.S. Cl. CPC A61B 5/4211; A61B 5/14539; A61B 5/0538; A61B 5/0022; A61B 5/14507; A61B 5/076; A61B 2560/0214; A61B 5/742; A61B 5/7475; A61B 2562/166; A61B 2560/0223; A61B 2562/162; A61B 5/1116

(57) ABSTRACT

An implantable capsule includes a capsule, an adsorption hole, a pH detector, two impedance sensors and a pose sensor. The capsule includes a first shell, a second shell and the first shell and the second shell defines an accommodating space. The adsorption hole is disposed at the first shell and attaches to the tube wall of the esophagus by the external force. The pH detector is disposed at the second shell to measure the pH value of the esophageal fluid. Two impedance sensors are disposed at the first shell and the second shell. The two impedance sensors measure the impedance of the esophageal fluid to determine the fluid direction of the esophageal fluid. The pose sensor is disposed at the second shell to determine the patient pose. By this configuration, lightweight and wireless gastroesophageal monitoring device and the long-time measurement of pH of the esophageal fluid may be achieved.

### CROSS-REFERENCE TO RELATED APPLICATION

This application claims priority from U.S. Provisional Patent Application No. 63/002,003, filed Mar. 30, 2020, the disclosures of which are hereby incorporated by reference in their entirety for all purposes.

### BACKGROUND OF THE INVENTION

### 1. Field of the Invention

The present invention relates to a gastroesophageal reflux disease determining system, and particularly to an implantable capsule in which the pH detector and the impedance sensor dispose for alleviating the discomfort of the patient and monitoring the esophagus and gastroesophageal reflux disease determining system thereof.

### 2. Description of the Related Art

The current gastroesophageal reflux disease monitoring device puts a tiny tube on the proper position of the esophagus through the nasal cavity to measure the pH value and the impedance in the esophagus, and further evaluates the composition, the direction and the pH value of the gastroesophageal reflux. Another terminal of the tube is connected to a small computer (approximately palm size). During the examination procedure, the patient records the eating time, the occurrence of time about the discomfort symptom and the sleeping time by the computer and the hard copy. After 24 hours monitoring, the patient will have to go the hospital to remove the tiny tube tomorrow. The physician would transmit the recorded data in the computer to another computer used by the physician. The physician would evaluates whether the patient has a gastroesophageal reflux disease or not based on the recorded data and the record paper of the patient for facilitating the subsequent treatment and therapy. Because the examination takes 24 hours, some patients are unable to tolerate the discomfort of the tube, thereby stopping examination or no discomfort symptom during examination. It influences the determination of the physician.

Accordingly, the inventor of the present invention has a gastroesophageal reflux disease determining system to overcome deficiencies in terms of current techniques so as to enhance the implementation and application in industries.

### SUMMARY OF THE INVENTION

In view of the aforementioned known issues, the purpose of the present invention is to provide an implantable capsule and gastroesophageal reflux disease determining system thereof to solve the problems found in the conventional techniques.

In order to achieve the objective, the present invention provides an implantable capsule including a capsule, an adsorption hole, a pH detector, two impedance sensors and a pose sensor. The capsule includes a first shell, a second shell and the first shell and the second shell defines an accommodating space. The adsorption hole is disposed at the first shell and attaches to the tube wall of the esophagus by the external force. The pH detector is disposed at the second shell to measure pH of the esophageal fluid. Two impedance sensors are disposed at the first shell and the second shell. The two impedance sensors measure the impedance of the esophageal fluid to determine the fluid direction of the esophageal fluid. The pose sensor is disposed at the second shell to determine the patient pose.

Optionally, the present invention further includes a printed circuit board disposed in the accommodating space and a wireless transceiver disposed on the printed circuit board. The wireless transceiver is electrically connected to the pH detector, two impedance sensors and the pose sensor to receive and transmit the pH value, the fluid direction of esophageal fluid and the patient pose.

Optionally, the present invention further includes a controller disposed on the printed circuit board and electrically connected to the pH detector, the two impedance sensors, the pose sensor and the wireless transceiver. The controller integrates the pH value, the fluid direction of esophageal fluid and the patient pose and digitalizes them into a digital information. The controller transmits the digital information through the wireless transceiver.

Optionally, the pH detector includes a set of electrodes disposed at the second shell and an operating circuit disposed on the printed circuit board and electrically connected to the set of electrodes and the controller. The set of electrodes is immersed in the esophageal fluid of esophagus to acquire an electrical signal, and the operating circuit enlarges the electrical signal and operates the pH value based on the enlarged electrical signal.

Optionally, the present invention further includes a power source disposed on the printed circuit board and electrically connected to the controller, the wireless transceiver, the pH detector, the two impedance sensors, the pose sensor to supply an electrical power.

In order to achieve the objective, the present invention provides a gastroesophageal reflux disease determining system including the aforementioned implantable capsule, a first electronic device and a cloud platform. The implantable capsule has a number and the wirelessly transmits the number. The first electronic device is wirelessly connected to the implantable capsule and receives the number, the pH value, the fluid direction of esophageal fluid and the patient pose. The cloud platform is internet-connected to the first electronic device. The first electronic device transmits the number to the cloud platform and the cloud platform performs an authentication process on the number to authenticate the implantable capsule. The cloud platform transmits a calibration parameter to the first electronic device after authenticating the number. The first electronic device calibrates the pH value based on the calibration parameter and calculates a gastroesophageal reflux disease index based on the calibrated pH value, the fluid direction of esophageal fluid and the patient pose.

Optionally, the first electronic device transmits the calibrated pH value, the fluid direction of esophageal fluid and the patient pose to the cloud platform.

Optionally, the first electronic device includes a database storing a reference pH value, a reference fluid direction of esophageal fluid and a reference patient pose. The first electronic device compares the calibrated pH value, the fluid direction of esophageal fluid and the patient pose with the reference pH value, the reference fluid direction of esophageal fluid and the reference patient pose to calculate the gastroesophageal reflux disease index.

Optionally, the present invention further includes a second electronic device wirelessly connected to the first electronic device or the implantable capsule. The second electronic device displays the calibrated pH value, the fluid direction of esophageal fluid and the patient pose.

Optionally, the present invention further includes a plurality of physiology measurement devices wirelessly connected to the first electronic device. Each physiology measurement device measures a physiological value.

Optionally, the database receives a time of occurrence of gastroesophageal reflux disease inputted by a user interface of the first electronic device. The first electronic device receives a time of occurrence of gastroesophageal reflux disease inputted by the user interface of the second electronic device. The first electronic device compares the time of occurrence of gastroesophageal reflux disease, the calibrated pH value, the fluid direction of esophageal fluid and the patient pose with the reference pH value, the reference fluid direction of esophageal fluid and the reference patient pose to calculate the gastroesophageal reflux disease index.

According to the above content, the present invention of the implantable capsule and gastroesophageal reflux disease determining system thereof can provide a lightweight and wireless gastroesophageal reflux disease monitoring device and be able to monitor the esophagus for ensuring whether the patient has the gastroesophageal reflux disease or not.

### BRIEF DESCRIPTION OF THE DRAWINGS

FIG. 1 depicts a schematic diagram of the gastroesophageal reflux disease determining system according to the first embodiment of the present invention.

FIG. 2 depicts a structure diagram of the implantable capsule according to the present invention.

FIG. 3 depicts a configuration diagram of the implantable capsule according to the present invention.

FIG. 4 depicts a schematic diagram of the gastroesophageal reflux disease determining system according to the second embodiment of the present invention.

FIG. 5 depicts a schematic diagram of the gastroesophageal reflux disease determining system according to the third embodiment of the present invention.

FIG. 6 depicts a schematic diagram of the gastroesophageal reflux disease determining system according to the fourth embodiment of the present invention.

### DESCRIPTION OF THE PREFERRED EMBODIMENTS

The following embodiments of the present invention are herein described in detail with reference to the accompanying drawings. These drawings show specific examples of the embodiments of the present invention. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and will not be interpreted in an idealized or overly formal sense, unless expressly so defined herein.

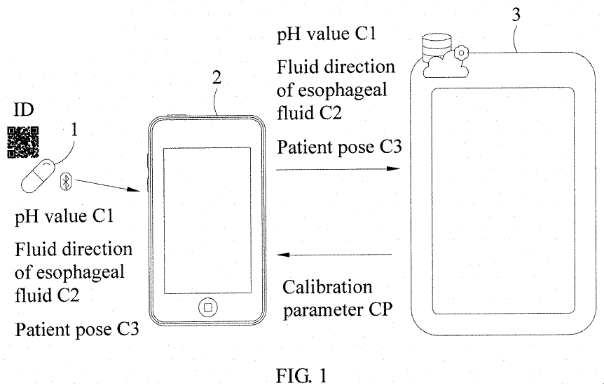

Please refer to FIG. 1, which depicts a schematic diagram of the gastroesophageal reflux disease determining system according to the first embodiment of the present invention. As shown by FIG. 1, the present invention of the gastroesophageal reflux disease determining system includes an implantable capsule 1, a first electronic device 2 and a cloud platform 3. The implantable capsule 1 is placed and fixed on the adjacent junction zone between the esophagus and the stomach through snout by the gastroscopy, and then the gastroscopy is taken out. The implantable capsule 1 is successfully placed in the patient's body. The implantable capsule 1 has the number ID, senses the pH value C1 of the esophageal fluid and the fluid direction of esophageal fluid C2, and detects the patient pose C3 of the patient. The implantable capsule 1 is wirelessly connected to the first electronic device 2 to wirelessly transmit the number ID, the pH value C1, the fluid direction of esophageal fluid C2 and the patient pose C3 to the first electronic device 2. The first electronic device 2 receives the number ID, the pH value C1, the fluid direction of esophageal fluid C2 and the patient pose C3. Or the first electronic device 2 scans the number ID of the package of the implantable capsule 1. The wireless connection between the implantable capsule 1 and the first electronic device 2 includes Wi-Fi, WiMax (Worldwide Interoperability for Microwave Access), ZigBee or Bluetooth. The wireless connection between the implantable capsule 1 and the first electronic device 2 may be the other better wireless connection, and is not limited to the scope stated by the present invention.

The first electronic device 2 is internet-connected to the cloud platform 3 and transmits the number ID to the cloud platform 3. The cloud platform 3 performs the authentication process AP on the number ID to authenticate the implantable capsule 1. The cloud platform 3 transmits a calibration parameter CP to the first electronic device 2 after authenticating the number ID. At the same time, the memory M records the authentication process AP performed by the cloud platform 3 and stores the authenticated number ID of the implantable capsule 1. The first electronic device 2 calibrates the pH value C1 based on the calibration parameter CP. The first electronic device 2 calculates the gastroesophageal reflux disease index based on the calibrated pH value C1, the fluid direction of esophageal fluid C2 and the patient pose C3. The first electronic device 2 transmits the calibrated pH value C1, the fluid direction of esophageal fluid C2, the patient pose C3 and the gastroesophageal reflux disease index to the cloud platform 3. Wherein, the first electronic device 2 may include the computer, the mobile device or the cell phone. The first electronic device 2 may be the other better electronic device provided with the wireless communication and the internet connection, and is not limited to the scope stated by the present invention.

Moreover, the first electronic device 2 has the symptom input interface (i.e. user interface) providing the patient to record the time point and the lasting time when the symptom of the gastroesophageal reflux disease and discomfort symptoms take place. The symptom of the gastroesophageal reflux disease includes the foreign body sensation during swallowing, chest pain, chronic cough or burn heart. The first electronic device 2 may provide the patient to input the personal health status. The personal health status includes the smoke condition, the height and weight, the medical situation or mental health condition. The personal health status may be the generation of the possible disease cause (e.g. mental stress, fatty and smoke). The first electronic device 2 calculates the correlation degree between the possible disease cause and the gastroesophageal reflux disease based on its database, and transmits the correlation degree between the possible disease cause and the gastroesophageal reflux disease to the cloud platform 3.

The first electronic device 2 compares the calibrated pH value C1, the fluid direction of esophageal fluid C2 and the patient pose C3 with the reference pH value, the reference fluid direction of esophageal fluid and the reference patient pose stored in the database of the first electronic device 2. The first electronic device 2 also records the time point when the symptom of the gastroesophageal reflux disease takes place, the lasting time of the calibrated pH value C1, the lasting time of the fluid direction of esophageal fluid C2 and the lasting time of the patient pose C3 and refers to the discomfort symptoms inputted by the patient. Hence, the first electronic device 2 calculates the gastroesophageal reflux disease index and the correlation degree between the possible disease cause and the gastroesophageal reflux disease. The doctor determines the health status of the patient based on the gastroesophageal reflux disease index and the correlation degree between the possible disease cause and the gastroesophageal reflux disease. The gastroesophageal reflux disease index is the probability of the gastroesophageal reflux disease.

The determination of the gastroesophageal reflux disease by the first electronic device 2 is described as follows: If the fluid direction of esophageal fluid C2 is from the stomach to the esophagus, the calibrated pH value C1 is lower than the first preset value (e.g. pH=6) and the patient pose C3 is the standing pose, the gastroesophageal reflux disease index calculated by the first electronic device 2 is higher (e.g. the gastroesophageal reflux disease index may be 85%). If the fluid direction of esophageal fluid C2 is from the esophagus to the stomach, the calibrated pH value C1 is higher than the first preset value and the patient pose C3 is the standing pose, the gastroesophageal reflux disease index calculated by the first electronic device 2 is lower (e.g. the gastroesophageal reflux disease index may be 20%). If the calibrated pH value C1 is higher than the reference value (pH=7), the gastroesophageal reflux disease index calculated by the first electronic device 2 is lower (e.g. the gastroesophageal reflux disease index may be 20%). If the fluid direction of esophageal fluid C2 is from the stomach to the esophagus, the calibrated pH value C1 is lower than the second preset value (e.g. pH=5) and the patient pose C3 is the prone position, the gastroesophageal reflux disease index calculated by the first electronic device 2 is higher (e.g. the gastroesophageal reflux disease index may be 85%). If the fluid direction of esophageal fluid C2 is from the esophagus to the stomach, the calibrated pH value C1 is higher than the second preset value and the patient pose C3 is the prone position, the gastroesophageal reflux disease index calculated by the first electronic device 2 is lower (e.g. the gastroesophageal reflux disease index may be 20%). If the calibrated pH value C1 is higher than the reference value (pH=7), the gastroesophageal reflux disease index calculated by the first electronic device 2 is lower (e.g. the gastroesophageal reflux disease index may be 20%). The doctor determine whether the patient has the gastroesophageal reflux disease or not based on the gastroesophageal reflux disease index and the correlation degree between the possible disease cause and the gastroesophageal reflux disease.

Besides, the first electronic device 2 may have a user interface, and the user interface displays the patient journal, the calibrated pH value C1, the fluid direction of esophageal fluid C2 and the patient pose C3. The implantable capsule 1 senses the calibrated pH value C1, the fluid direction of esophageal fluid C2 and the patient pose C3 based on the sampling frequency, and the first electronic device 2 records the calibrated pH value C1, the fluid direction of esophageal fluid C2 and the patient pose C3. Afterward the first electronic device 2 draws the curve of the calibrated pH value C1 and reveals the curve of the calibrated pH value C1 to the patient by its user interface. The first electronic device 2 is internet-connected to the hospital platform. The first electronic device 2 has related information about the treatment of patient by the hospital (inclusive of the medicine information and the registration information), and the user interface of the first electronic device 2 reminds the patient to take medicine and the registration time.

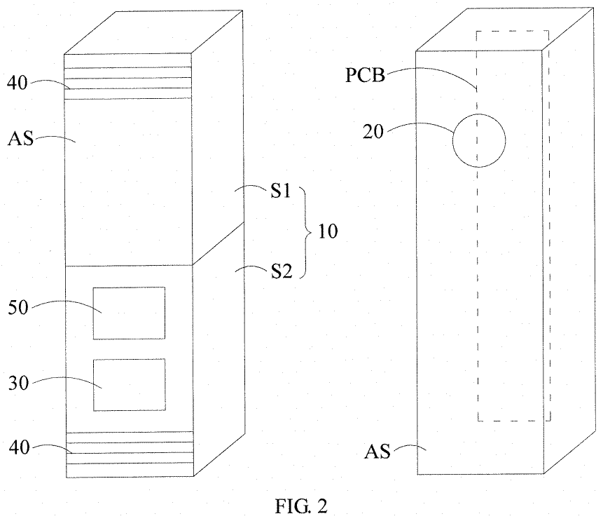

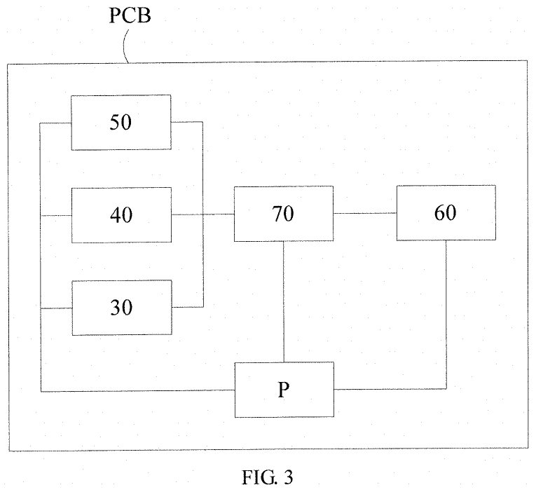

Please refer to FIG. 2 and FIG. 3, which depicts a structure diagram of the implantable capsule according to the present invention and a configuration diagram of the implantable capsule according to the present invention. As shown by FIG. 2 and FIG. 3, the present invention of the implantable capsule 1 includes a capsule 10, an adsorption hole 20, a pH detector 30, two impedance sensors 40 and a pose sensor 50. The capsule 10 includes a first shell S1 and a second shell S2. The first shell S1 and the second shell S2 defines an accommodating space AS. Specifically, when the first shell S1 and the second shell S2 are engaged with each other, the interior of the first shell S1 and the second shell S2 forms the accommodating space AS. The adsorption hole 20 is disposed at the first shell S1 and attaches to the tube wall of the esophagus by the external force. For example, the implantable capsule 1 is placed at 6 cm above the adjacent junction zone between the esophagus and the stomach by the gastroscopy, and the gastroscopy applies the stress to the adsorption hole 20. Hereby, the adsorption hole 20 attaches to the tube wall of the esophagus and the implantable capsule 1 is fixed. The pH detector 30 is disposed at the second shell S2 to measure the pH value of the esophageal fluid. Two impedance sensors 40 are disposed at the first shell S1 and the second shell S2. Two impedance sensors 40 measure the impedance of the esophageal fluid to determine the fluid direction of the esophageal fluid C2. The pose sensor 50 is disposed at the second shell S2 to determine the patient pose C3.

The present invention further includes the printed circuit board PCB, the wireless transceiver 60, the controller 70 and the power source P. The printed circuit board PCB is disposed in the accommodating space AS. The wireless transceiver 60, the controller 70, the operating circuit and the power source P is disposed on the printed circuit board PCB. The controller 70 is electrically connected to the pH detector 30, two impedance sensors 40, the pose sensor 50 and the wireless transceiver 60. Two impedance sensors 40 and the pose sensor 50 are connected to the controller through a plurality of wires. The power source P is electrically connected to the controller 70, the wireless transceiver 60, the pH detector 30, two impedance sensors 40 and the pose sensor 50. The power source P supplies the electrical power to the controller 70, the wireless transceiver 60, the pH detector 30, two impedance sensors 40 and the pose sensor 50.

In one embodiment, there is no controller 70 on the printed circuit board PCB, and the wireless transceiver 60 is electrically connected to the pH detector 30, two impedance sensors 40, the pose sensor 50 to receive the pH value C1, the fluid direction of esophageal fluid C2 and the patient pose C3. The wireless transceiver 60 receives and wirelessly transmits the pH value C1, the fluid direction of esophageal fluid C2 and the patient pose C3 to the first electronic device 2 (as illustrated by FIG. 2). In another embodiment, there is the controller 70 on the printed circuit board PCB, and the controller 70 receives and integrates the pH value C1, the fluid direction of esophageal fluid C2 and the patient pose C3. The controller 70 digitalizes the pH value C1, the fluid direction of esophageal fluid C2 and the patient pose C3 into the digital information and transmits the digital information to the first electronic device 2 through the wireless transceiver 60 (as illustrated by FIG. 2).

In one embodiment, the pH detector 30 includes the set of electrodes and the operating circuit. The set of electrodes is disposed at the second shell to be immersed in the esophageal fluid of esophagus. The operating circuit is electrically connected to the set of electrodes. The set of electrodes is immersed in the esophageal fluid of esophagus to generate a voltage, and the operating enlarges the voltage and operates the pH value based on the enlarged voltage. In another embodiment, the pH detector 30 includes the conductive film and the operating circuit. The conductive film is disposed at the second shell to contact the esophageal fluid of esophagus. The operating circuit is electrically connected to the conductive film. The conductive film contacts the esophageal fluid of esophagus to generate a current, and the operating enlarges the current and operates the pH value based on the enlarged current.

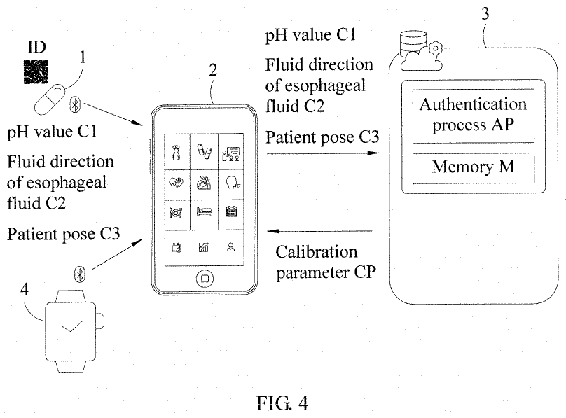

Please refer to FIG. 4, which depicts a schematic diagram of the gastroesophageal reflux disease determining system according to the second embodiment of the present invention. As shown by FIG. 4, the present invention of the gastroesophageal reflux disease determining system includes an implantable capsule 1, a first electronic device 2 and a cloud platform 3. The second embodiment of the present invention is similar to the first embodiment of the present invention, and the similarity between the first embodiment and the second embodiment is not repeated here. However, there is a difference between the first embodiment and the second embodiment: the present invention further includes a second electronic device 4 and the first electronic device 2 includes a database. The database previously stores the reference pH value, the reference fluid direction of esophageal fluid and the reference patient pose corresponding to the gastroesophageal reflux disease. The first electronic device 2 compares the calibrated pH value C1, the fluid direction of esophageal fluid C2 and the patient pose C3 with the reference pH value, the reference fluid direction of esophageal fluid and the reference patient pose to calculate the gastroesophageal reflux disease index. The gastroesophageal reflux disease index is the related degree between the calibrated pH value C1, the fluid direction of esophageal fluid C2 and the patient pose C3 and the reference pH value, the reference fluid direction of esophageal fluid and the reference patient pose. The gastroesophageal reflux disease index is high, that is to say that the probability of the condition that the patient has the gastroesophageal reflux disease is high. The gastroesophageal reflux disease index is low, that is to say that the probability of the condition that the patient has the gastroesophageal reflux disease is low. The second electronic device 4 is wirelessly connected to the first electronic device 2. The second electronic device 4 reveals the calibrated pH value C1, the fluid direction of esophageal fluid C2 and the patient pose C3. The patient can manipulate the user interface of the second electronic device 4 to input the time point when the symptom of the gastroesophageal reflux disease takes place. The second electronic device 4 transmits the time point when the symptom of the gastroesophageal reflux disease takes place to the first electronic device 2 for reference to calculate the gastroesophageal reflux disease index. Wherein, the second electronic device 4 may be the wearable electronic device or the smart watch. The second electronic device 4 may be the other better electronic device, and is not limited to the scope stated by the present invention.

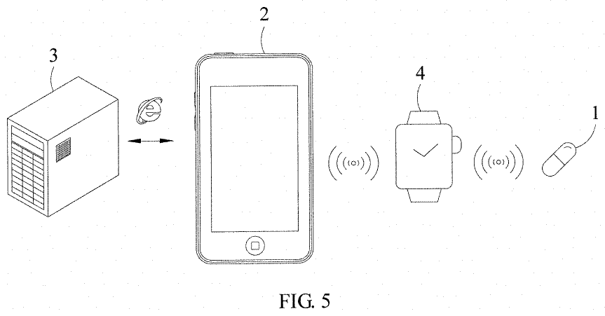

Please refer to FIG. 5, which depicts a schematic diagram of the gastroesophageal reflux disease determining system according to the third embodiment of the present invention. As shown by FIG. 5, the present invention of the gastroesophageal reflux disease determining system includes an implantable capsule 1, a first electronic device 2 and a cloud platform 3. The third embodiment of the present invention is similar to the second embodiment of the present invention, and the similarity between the second embodiment and the third embodiment is not repeated here. However, there is a difference between the second embodiment and the third embodiment: the second electronic device 4 is wirelessly connected to the implantable capsule 1 and the first electronic device 2. The second electronic device 4 receives the number ID, the pH value C1, the fluid direction of esophageal fluid C2 and the patient pose C3 and transmits them to the first electronic device 2. The first electronic device 2 transmits the number ID to the cloud platform 3 to perform the authentication process (as illustrated by FIG. 1). The first electronic device 2 acquires the calibration parameter CP after authenticating the number ID to calibrate the pH value C1. The first electronic device 2 calculates the gastroesophageal reflux disease index based on the calibrated pH value C1, the fluid direction of esophageal fluid C2 and the patient pose C3. Furthermore, the second electronic device 4 detects the temperature and the heart rate of the patient to record, and the second electronic device 4 integrates the temperature, the heart rate of the patient, the calibrated pH value C1, the fluid direction of esophageal fluid C2 and the patient pose C3 for calculating the gastroesophageal reflux disease index.

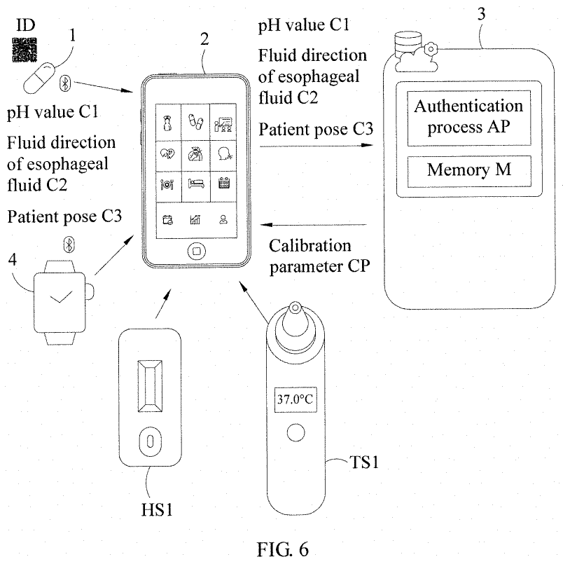

Please refer to FIG. 6, which depicts a schematic diagram of the gastroesophageal reflux disease determining system according to the fourth embodiment of the present invention. As shown by FIG. 5, the present invention of the gastroesophageal reflux disease determining system includes an implantable capsule 1, a first electronic device 2 and a cloud platform 3. The fourth embodiment of the present invention is similar to the second embodiment of the present invention, and the similarity between the second embodiment and the fourth embodiment is not repeated here. However, there is a difference between the second embodiment and the fourth embodiment: the present invention further includes a plurality of physiology measurement devices. The first electronic device 2 is wirelessly connected to each physiology measurement device. Each physiology measurement device measure the physiology parameter. For example, the plurality of physiology measurement devices includes the temperature detecting device TS1 and the heart rate detecting device HS1. The temperature detecting device TS1 measures the temperature of the patient. The heart rate detecting device HS1 measures the heart rate of the patient. The first electronic device 2 compares the temperature and the heart rate with the reference temperature and the reference heart rate to calculate the gastroesophageal reflux disease index. The physiology measurement device may be the sphygmomanometer or the respiratory rate measurement device, and is not limited to the scope stated by the present invention.

According to the above content, the present invention of the implantable capsule and gastroesophageal reflux disease determining system thereof can provide a lightweight and wireless gastroesophageal reflux disease monitoring device and be able to monitor the esophagus for ensuring whether the patient has the gastroesophageal reflux disease or not.

The present invention may be realized in different forms and should not be construed as being limited to the embodiments mentioned herein. It is to be understood that many other possible modifications and variations can be made by persons having ordinary skill in the art without departing from the spirit and scope of the invention as hereinafter claimed.

What is claimed is:

1. An implantable capsule comprising:
a capsule, comprising a first shell and a second shell and defining an accommodating space by the first shell and the second shell;
an adsorption hole disposed at the first shell and that attaches to a tube wall of an esophagus by an external force;
a pH detector disposed at the second shell to measure a pH value of an esophageal fluid;
two impedance sensors disposed at the first shell and the second shell and measuring an impedance of the esophageal fluid to determine a fluid direction of esophageal fluid; and
a pose sensor disposed at the second shell to determine a patient pose.

2. The implantable capsule of claim 1, further comprising a printed circuit board disposed in the accommodating space and a wireless transceiver disposed on the printed circuit board; the wireless transceiver electrically connected to the pH detector, the two impedance sensors, and the pose sensor to receive and transmit the pH value, the fluid direction of esophageal fluid, and the patient pose.

3. The implantable capsule of claim 2, further comprising a controller disposed on the printed circuit board and electrically connected to the pH detector, the two impedance sensors, the pose sensor, and the wireless transceiver; the controller integrating the pH value, the fluid direction of esophageal fluid, and the patient pose and digitalizing them into a digital information; the controller transmitting the digital information through the wireless transceiver.

4. The implantable capsule of claim 3, wherein the pH detector comprises a set of electrodes disposed at the second shell and an operating circuit disposed on the printed circuit board and electrically connected to the set of electrodes and the controller, the set of electrodes configured to be immersed in the esophageal fluid of the esophagus to acquire an electrical signal, the operating circuit enlarging the electrical signal and operating the pH value based on the enlarged electrical signal.

5. The implantable capsule of claim 3, further comprising a power source disposed on the printed circuit board and electrically connected to the controller, the wireless transceiver, the pH detector, the two impedance sensors, and the pose sensor to supply an electrical power.

6. The gastroesophageal reflux disease determining system, comprising:
an implantable capsule according to claim 1 and having a number, and wirelessly transmitting the number;
a first electronic device wirelessly connected to the implantable capsule and receiving the number, the pH value, the fluid direction of esophageal fluid, and the patient pose; and
a cloud platform internet-connected to the first electronic device, the first electronic device transmitting the number to the cloud platform, the cloud platform performing an authentication process on the number to authenticate the implantable capsule, the cloud platform transmitting a calibration parameter to the first electronic device after authenticating the number, the first electronic device calibrating the pH value based on the calibration parameter and calculating a gastroesophageal reflux disease index based on the calibrated pH value, the fluid direction of esophageal fluid, and the patient pose.

7. The gastroesophageal reflux disease determining system of claim 6, wherein the first electronic device is configured to transmit the calibrated pH value, the fluid direction of esophageal fluid, and the patient pose to the cloud platform.

8. The gastroesophageal reflux disease determining system of claim 6, wherein the first electronic device comprises a database storing a reference pH value, a reference fluid direction of esophageal fluid, and a reference patient pose; the first electronic device comparing the calibrated pH value, the fluid direction of esophageal fluid, and the patient pose with the reference pH value, the reference fluid direction of esophageal fluid, and the reference patient pose to calculate the gastroesophageal reflux disease index.

9. The gastroesophageal reflux disease determining system of claim 8, further comprising a second electronic device wirelessly connected to the first electronic device or the implantable capsule, the second electronic device displaying the calibrated pH value, the fluid direction of esophageal fluid, and the patient pose.

10. The gastroesophageal reflux disease determining system of claim 9, wherein the database receiving a time of occurrence of discomfort symptoms inputted by a user interface of the first electronic device, the first electronic device receiving a time of occurrence of discomfort symptoms inputted by the user interface of the second electronic device, the first electronic device comparing the time of occurrence of discomfort symptoms, the calibrated pH value, the fluid direction of esophageal fluid, and the patient pose with the reference pH value, the reference fluid direction of esophageal fluid, and the reference patient pose to calculate the gastroesophageal reflux disease index.
